(19) United States
(12) Patent Application Publication
(10) Pub. No.: US 2026/0232289 A1
(43) Pub. Date: Aug. 13, 2026

(54) ULTRASOUND ERCP GUIDEWIRE

(71) Applicant: BOSTON SCIENTIFIC SCIMED, INC., Maple Grove, MN (US)

(72) Inventors: Daniel BRIONES MEDINA, San José (CR); Hannah Catherine ANDERSEN, Brookline, MA (US); Laura Emily SABBAN, Norton, MA (US); Juan Pablo ORTIZ GARCIA, Heredia (CR); Daniel Joseph FAULKNER, Framingham, MA (US); Nathan Michael TENNEY, Leominster, MA (US)

(21) Appl. No.: 19/447,756

(22) Filed: Jan. 13, 2026

Related U.S. Application Data
(60) Provisional application No. 63/750,023, filed on Jan. 27, 2025.

(51) Int. Cl. A61B 8/08; A61B 1/00; A61B 1/24; A61B 8/00

(52) U.S. Cl. CPC A61B 8/085; A61B 1/00006; A61B 1/00124; A61B 1/00154; A61B 1/24; A61B 8/5207

(57) ABSTRACT

A system is for accessing a biliary and pancreatic duct. The system includes a guidewire with a shaft configured to be inserted through a channel of an endoscope to a target location within a duct anatomy. The shaft extends longitudinally from a proximal end to a distal end. The distal end includes a transducer configured to produce signals for generating an ultrasound image. The system also includes an electronic connector configured to connect the transducer to a processor configured to analyze electric signals transmitted from the transducer to generate the image.

### PRIORITY CLAIM

The present disclosure claims priority to U.S. Provisional Patent Application Ser. No. 63/750,023 filed Jan. 27, 2025; the disclosure of which is incorporated herewith by reference

### BACKGROUND

Endoscopic retrograde cholangiopancreatology (ERCP) is a procedure used to diagnose and/or treat problems in the liver, gallbladder, bile, and pancreas. In particular, an endoscope may be guided through a patient's gastrointestinal tract—e.g., through a mouth and throat, down the esophagus, stomach, and the duodenum. A small tube may then be inserted through the endoscope into the biliary ducts, to inject a contrast dye which facilitates visualization of the area via X-ray. This allows a healthcare provider to identify any issues such as, for example, blockages in the ducts—e.g., gallstones, tumors, scar tissue, etc. In some cases, the identified issues may also be treated via the endoscope. In these cases, an ERCP guidewire may be inserted through the endoscope to navigate the pancreaticobiliary ducts and introduce instruments for diagnostic or therapeutic interventions. Current ERCP guidewires, however, may face navigational challenges as it is often difficult to maneuver the guidewire through complex ductal anatomy as desired.

### SUMMARY

The present disclosure relates to a system for accessing a biliary and pancreatic duct. The system includes a guidewire including a shaft configured to be inserted through a channel of an endoscope to a target location within a duct anatomy, the shaft extending longitudinally from a proximal end to a distal end, the distal end including a transducer configured to produce signals for generating an ultrasound image. The system also includes an electronic connector configured to connect the transducer to a processor configured to analyze electric signals transmitted from the transducer to generate the image.

In an embodiment, the system further includes a rotator configured to rotate the transducer about a longitudinal axis of the guidewire so that the transducer is configured to generate a 360-degree ultrasound image.

In an embodiment, the transducer includes a transducer configured to generate a 360-degree ultrasound image.

In an embodiment, the transducer includes a piezoelectric transducer.

In an embodiment, the distal end of the shaft includes a hydrophilic coating.

In an embodiment, a proximal portion of the shaft of the guidewire includes a telescope mechanism movable between an expanded configuration and a contracted configuration.

In an embodiment, the telescope mechanism includes a plurality of telescoping elements, each of the telescoping elements configured to be sequentially received within an immediately adjacent one of the telescoping elements as the telescope mechanism is moved from the expanded configuration toward the contracted configuration.

In an embodiment, the telescope mechanism includes markings indicating a length of contraction of the guidewire.

In an embodiment, the electronic connector includes a PCBA board.

In addition, the present disclosure relates to a guidewire device which includes a shaft configured to be inserted through a channel of an endoscope to a target location within a duct anatomy, the shaft extending from a proximal end to a distal end including a transducer configured to emit an ultrasonic energy to a surrounding tissue.

In an embodiment, the transducer receives reflected echoes and converts them into electrical signals to generate an ultrasound image.

In an embodiment, the device further includes a rotator at the proximal end of the shaft, the rotator configured to rotate the transducer about a longitudinal axis of the device so that the transducer is configured to generate a 360-degree ultrasound image.

In an embodiment, the device further includes a telescope mechanism along a proximal portion of the shaft, the telescope mechanism movable between an expanded configuration and a contracted configuration to measure a length of contraction of the shaft.

In an embodiment, the telescope mechanism includes a plurality of telescoping elements, each of the telescoping elements configured to be sequentially received within an immediately adjacent one of the telescoping elements as the telescope mechanism is moved from the expanded configuration toward the contracted configuration.

In an embodiment, the shaft includes a plurality of the transducers extending along a distal portion thereof, the plurality of the transducer configured to deliver ultrasonic energy for dissolving a biliary stone.

In addition, the present disclosure relates to a method for treating a duct anatomy during an endoscopic retrograde cholangiopancreatology. The method includes inserting a guidewire through a channel of an endoscope to a target location within the duct anatomy and navigating the guidewire to the target location under ultrasound image guidance provided via a transducer at a distal end of the guidewire, which emits ultrasonic energy to a surrounding tissue to generate an ultrasound image.

In an embodiment, the method further includes positioning the distal end of the guidewire in alignment with a distal end of an obstruction within the target location of the duct anatomy; and retracting a distal portion of the guidewire toward a proximal end of the guidewire until the distal end of the guidewire is in alignment with a proximal end of the obstruction within the target location to determine a length of the obstruction.

In an embodiment, the retracting the distal portion of the guidewire toward the proximal end of the guidewire includes moving a telescope mechanism from an expanded configuration toward a contracted configuration.

In an embodiment, the telescope mechanism includes a plurality of telescoping elements configured to be sequentially received within an immediately adjacent one of the telescoping elements as the telescope mechanism is moved from the expanded configuration toward the contracted configuration.

In an embodiment, the transducer is rotatable about a longitudinal axis of the guidewire via a rotator at a proximal end of the guidewire to generate a 360-degree ultrasound image.

### BRIEF DESCRIPTION

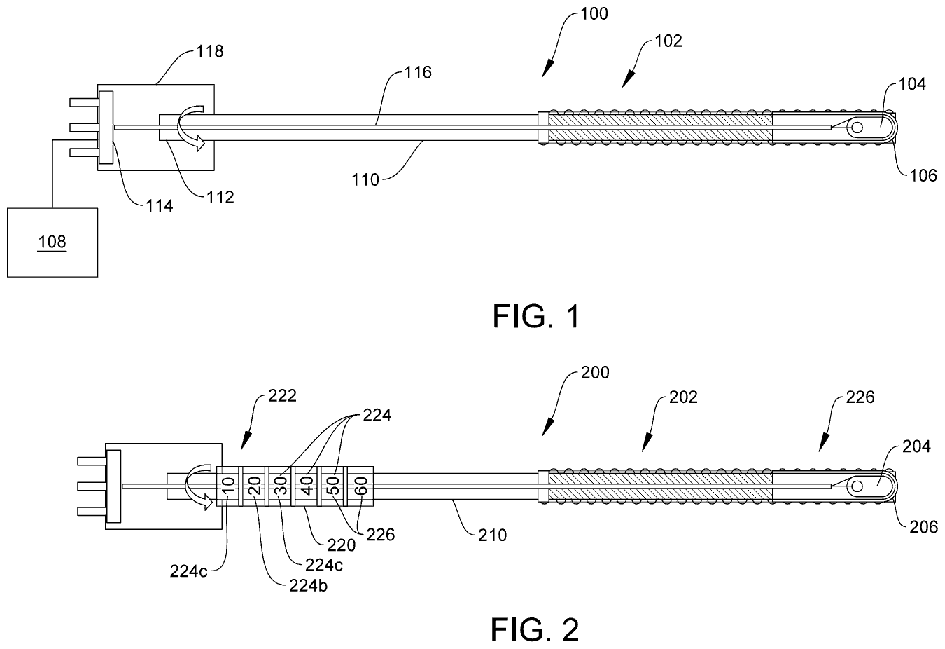

FIG. 1 shows a schematic drawing of a system according to an exemplary embodiment of the present disclosure;

FIG. 2 shows a schematic drawing of a system according to a further exemplary embodiment of the present disclosure;

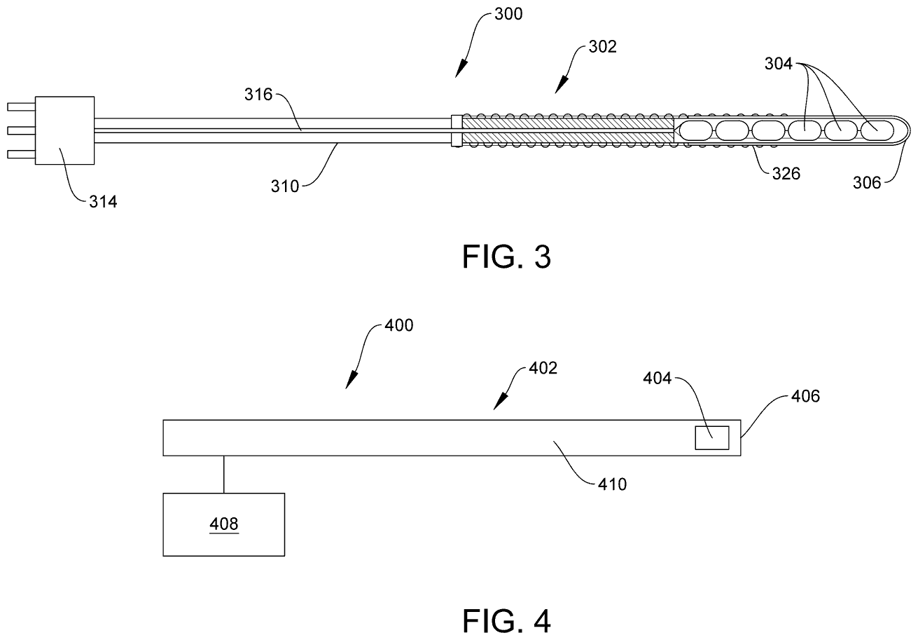

FIG. 3 shows a schematic drawing of a system according to another exemplary embodiment of the present disclosure; and

FIG. 4 shows a schematic drawing of a system according to yet another exemplary embodiment of the present disclosure.

### DETAILED DESCRIPTION

The present disclosure may be further understood with reference to the following description and appended drawings, wherein like elements are referred to with the same reference numerals. The present disclosure relates to endoscopic systems and procedures and, in particular, relates to a guidewire configured to access and maintain access to the bile and pancreatic ducts during an ERCP procedure, to allow for passage and exchange of instruments. Exemplary embodiments describe guidewires including features configured to facilitate navigation of the guidewire through biliary and/or pancreatic ducts for diagnosis and/or treatment thereof. Although the exemplary embodiments are specifically described with respect to an ERCP procedure, it will be understood by those of skill in the art that the exemplary systems may be utilized in any of a variety of procedures in which it is desired to navigate a guidewire through similarly complex anatomy. It should also be noted that the terms “proximal” and “distal,” as used herein, are intended to refer to a direction toward (proximal) and away from (distal) a user of the device (e.g., physician).

As shown in FIG. 1, a system 100 according to an exemplary embodiment of the present disclosure comprises a guidewire 102 including a transducer 104 at a distal end 106 thereof for facilitating navigation of the guidewire 102 through a duct (e.g., biliary, pancreatic) of a patient under ultrasound image guidance during, for example, an ERCP procedure. According to an exemplary embodiment, the guidewire 102 is configured to be passed through a channel of an endoscope (not shown), which has been inserted through a gastrointestinal (GI) tract and positioned within a portion of the small intestine (e.g., duodenum), so that the guidewire 102 may extend distally therefrom to be further navigated into and through biliary and/or pancreatic ducts to a target location therewithin. The guidewire 102 provides access to the target location so that treatment and/or diagnostic instruments may be passed over to guidewire 102 to reach target locations as would be understood by those skilled in the art. The guidewire 102 may be connected to a computing device 108, which may include, for example, a processor, display, and memory.

The transducer 104 may include, for example, a piezoelectric transducer (PZT) which emits mechanical soundwaves that reflect off of body tissue. As would be understood by those skilled in the art. the characteristics of the tissue or other substances from which these waves are reflected changes characteristics of these soundwaves in a manner which may be analyzed by the processor of the computing device 108 to generate an ultrasound image of the surroundings on the display of the computing device 108. This permits the guidewire 102 to be navigated to the target location, through even complex ductal anatomy, under ultrasound image guidance.

The guidewire 102 includes a shaft 110 extending longitudinally from a proximal end 112 to the distal end 106. In an exemplary embodiment, the shaft 110 is formed of a material having a stiffness configured to facilitate advancement of the guidewire 102 through the GI tract (via the endoscope) and the ductal anatomy. The shaft 110, however, also has sufficient flexibility to be navigated through the anatomy to the target location. In an exemplary embodiment, the shaft 110 may be formed of a material such as, for example, nitinol and/or stainless steel, and may include a hydrophilic coating along at least the distal end 106 to reduce surface friction and increase lubricity as the shaft 110 is guided to the target location within the ductal anatomy. In an exemplary embodiment, the shaft 110 is sized, shaped, and configured to be received within narrow ductal anatomy and, in one example, may have an outer diameter of up to 40 microns.

As described above, the guidewire 102 includes the transducer 104 at the distal end 106 of the shaft 110. The transducer 104 may be mounted within the distal end 106. In an exemplary embodiment, the transducer 104 is configured as a PZT (lead zirconate titanate) transducer which converts electrical signals into ultrasonic waves to generate ultrasound images. As would be understood by those of skill in the art, the PZT transducer can both emit ultrasound pulses and receive the reflected echoes, converting them back into electrical signals which are processed (e.g., via the computing device 108) to generate an ultrasound image.

The computing device 108 may include any of a variety of computers and/or other processing devices including, for example, a processor configured to analyze the signals received from the transducer 104 and a display for displaying the generated ultrasound image. The computing device 108 may further include a memory configured to include instructions for analyzing the electric signal from the transducer 104 and may include, for example, a non-transitory computer readable storage medium including instructions that are executable via the processor. The processor may be configured to execute computer-executable instructions for operations from applications stored in the memory of the processor to provide functionalities to the system 100. It will be understood by those of skill in the art that although the system 100 is shown as including a single computing device 108, the functionalities described with respect to the system 100 may be achievable via a modular component connected thereto or via more than one computing device 108.

Transfer of signals from the transducer 104 to the computing device 108 may be facilitated via an electronic connector 114 configured as, for example, a PCBA board, at the proximal end 112 of the shaft 110. The transducer 104 may be connected to the electronic connector 114 via a cable 116 (e.g., coaxial cable) extending along a length of the shaft 110 from the transducer 104 to the electronic connector 114. The cable 116 is configured to transmit signals from the transducer 104 to the computing device 108 via the electronic connector 114.

In an exemplary embodiment, the guidewire 102 also includes a rotator 118 at the proximal end 112 of the shaft 110. The rotator 118 is configured to rotate the shaft 110 and/or the transducer 104 to provide a 360-degree ultrasound image. The rotator 118 may include, for example, a rotational motor configured to apply torsional force to the shaft 110 and/or the transducer 104. In an alternate embodiment, rather than having a rotator 118, the guidewire 102 includes a plurality of the transducers 104 which together, are capable of providing a 360-degree image.

According to an exemplary method utilizing the system 100, the guidewire 102 is insertable through a channel of an endoscope to be navigated distally past a distal end thereof to a target location within a biliary or pancreatic duct. The transducer 104 at the distal end 106 of the shaft 110 of the guidewire 102 facilitates generation of an ultrasound image so that the guidewire 102 is navigable through the patient ducts to the site of, for example, an obstruction or damaged tissue requiring further treatment and/or diagnosis, via the ultrasound image. As described above, signals from the transducer 104 are analyzed via the computing device 108 to form an image displayed on a display thereof.

According to a further exemplary embodiment, as shown in FIG. 2, a system 200 may be substantially similar to the system 100 comprising a guidewire 202 including a transducer 204 at a distal end 206 of a shaft 210 thereof for facilitating navigation of the guidewire 202 through, for example, biliary and/or pancreatic ducts, under ultrasound image guidance. The guidewire 202, however, further comprises a telescope mechanism 220 along a proximal portion 222 thereof. The telescope mechanism 220 is configured to extend and contract a length of the proximal portion 222 and, in an exemplary embodiment, may be utilized to determine a length of an obstruction and/or damaged tissue section of a duct during an ERCP procedure, as will be described in further detail below.

The telescope mechanism 220 includes a plurality of tubular elements 224 movably connected to one another so that the telescope mechanism is movable between an expanded configuration and a contracted configuration. In an exemplary embodiment, each of the tubular elements 224 is sequentially slidable within an immediately adjacent one of the tubular elements 224. The telescope mechanism 220 is movable from the expanded configuration toward the contracted configuration by retracting a distal portion 226 of the guidewire 202 toward the proximal portion 222. As the guidewire 202 is contracted, a first one 224a of the tubular elements 224 is slidably received within an immediately adjacent second one 224b of the tubular elements 224, the second one 224b of the tubular elements 224 is slidably received within an immediately adjacent the third one 224c of the tubular elements 224, etc.

The tubular elements 224 may be sequentially received within one another so that a user (e.g., physician or other medical provider) may determine a length of contraction of the guidewire 202. In other words, the second one 224b of the tubular elements 224 will not be received within the third one 224c of the tubular elements 224 until the first one 224a of the tubular elements 224 has been completely received within the second one 224b of the tubular elements 224. The proximal portion 222 and/or the telescope mechanism 220 may include markings 225 thereon so that a length of retraction may be identified via the markings 225. The markings 225 may include, for example, numbers, lines, etc. It will be understood by those of skill in the art, however, that the markings 225 may include any of a variety of configurations so long as the markings provide a visual indication of the length of retraction.

According to an exemplary method utilizing the system 200, the guidewire 202 may be inserted to a target location within a duct utilizing ultrasound image guidance, substantially as described above with respect to the system 100. The guidewire 202 is inserted to the target location, in its fully expanded configuration, so that upon insertion of the guidewire 202 to the target location, the guidewire 202 may be used to measure and/or determine a length of an obstruction and/or damaged portion of tissue. In particular, according to an exemplary embodiment, the guidewire 202 is inserted through the obstruction/damaged tissue so that the distal end 206 is aligned with a distal end of the obstruction/damaged tissue. The guidewire 202 is then retracted while holding more proximal portions of the guidewire 202 in place, by drawing the distal portion 226 proximally toward the proximal portion 222, until the distal end 206 of the guidewire 202 is aligned with a proximal end of the obstruction/damaged tissue.

As the guidewire 202 is retracted, the telescope mechanism 220 moves from the fully expanded configuration toward the contracted configuration. As described above, the tubular elements 224 of the telescope mechanism 220 are configured to be sequentially received within one another so that the user may readily a length of contraction of the telescope mechanism 220. Thus, an extent and/or length of contraction of the telescope mechanism 220 corresponds to a length of the obstruction/damaged tissue. Identifying a length of the obstruction/damaged tissue permits a user to determine, for example, a length of a stent required to treat the duct during an ERCP procedure.

According to another exemplary embodiment, as shown in FIG. 3, a system 300 may be substantially similar to the system 100 comprising a guidewire 302 configured to be inserted through a channel of an endoscope to a target location within a biliary and/or pancreatic duct. Similarly to the guidewire 102, the guidewire 302 includes a shaft 310 having a transducer 304 (e.g., a piezoelectric PZT transducer) at a distal end 306 thereof. Rather than providing an ultrasound image or in addition to providing such images, the guidewire 302, includes a plurality of the transducers 304 configured to utilize ultrasound waves to treat an obstruction such as, for example, a stone.

The transducers 304 are positioned along a distal portion 326 of the shaft 310 of the guidewire 302. The guidewire 302 may include any number of the transducers 304 positioned along a length of the distal portion 326. A number of the transducers 304 may be selected based on, for example, a size of the stone to be treated. For example, the larger the stone, the larger a number of the transducers 304. In addition, a frequency of the transducers 304 may be selected based on a desired treatment. As indicated above, the transducers 304 may be supplemented by a transducer such as the transducer 104 configured for imaging purposes.

Similarly to the system 100, the system 300 includes an electronic connector 314 configured to connect the transducers 304 to a computing device (not shown) via a cable 316. The electronic connector 314 may be configured as a PCBA board facilitating communication with the computing device such that the computing device may be utilized to control delivery of the acoustic energy via the transducers 304.

According to an exemplary method utilizing the system 300, the guidewire 302 may be inserted through a channel of an endoscope to a target location within a duct. The distal end 306 may be placed against and/or inserted into the stone or other obstruction to be treated so that the transducers 304 deliver acoustic energy to the stone, dissolving the stone.

According to yet another exemplary embodiment, as shown in FIG. 4, a system 400 may be substantially similar to the system 100 described above, comprising a guidewire 402 configured to be inserted through a channel of an endoscope to a target location within a biliary and/or pancreatic duct. The guidewire 402, however, includes a sensor 404 at a distal end 406 of a shaft 410 thereof so that, as the guidewire is inserted through the ductal anatomy, the sensor 404 generates a map of the biliary system. The sensor 404 may utilize, for example, MicroFidelity (MiFi) sensor technology to generate a three-dimensional map of the biliary tree.

IntellaTip MiFi™ technology (Boston Scientific Corp., MA) employs miniaturized electrodes at the catheter tip to capture highly localized electrical signals, providing precise mapping of tissue characteristics, providing precise mapping of tissue characteristics. In an exemplary embodiment, this MiFi technology may be similarly utilized via an ERCP guidewire. For example, the sensor 404 of the guidewire 402 may be configured as a sensor array that is embedded at a tip of the distal end 406 to enable real-time measurement of electrical impedance or conductivity within the biliary and pancreatic ducts. It will be understood by those of skill in the art that this innovation would enhance the guidewire's ability to detect and characterize obstructions, strictures, or other ductal anomalies, significantly improving navigation and facilitating the accurate placement of devices such as stents or balloons. It will also be understood by those of skill in the art that this integration of MiFi technology would also enhance maneuverability through complex or challenging ductal anatomies, particularly in cases involving tight strictures or distorted anatomy

Similarly to the system 100, the system 400 comprises a computing device 408, a processor of which is configured to receive data from the sensor 404 to analyze the sensor data and generate a biliary model via biliary mapping. The generated three-dimensional model may be displayed on a display of the computing device 408 which, in an exemplary embodiment, may include a user interface via which a user may input user selections and/or settings related to the mapped biliary system.

It will be appreciated by those skilled in the art that changes may be made to the embodiments described above without departing from the inventive concept thereof. It should further be appreciated that structural features and methods associated with one of the embodiments can be incorporated into other embodiments. It is understood, therefore, that this invention is not limited to the particular embodiment disclosed, but rather, modifications are also covered within the scope of the present invention as defined by the appended claims.

1-15. (canceled)

16. A system for accessing a biliary and pancreatic duct, comprising:
a guidewire including a shaft configured to be inserted through a channel of an endoscope to a target location within a duct anatomy, the shaft extending longitudinally from a proximal end to a distal end, the distal end including a transducer configured to produce signals for generating an ultrasound image; and
an electronic connector configured to connect the transducer to a processor configured to analyze electric signals transmitted from the transducer to generate the image.

17. The system of claim 16, further comprising:
a rotator configured to rotate the transducer about a longitudinal axis of the guidewire so that the transducer is configured to generate a 360-degree ultrasound image.

18. The system of claim 16, wherein the transducer includes a transducer configured to generate a 360-degree ultrasound image.

19. The system of claim 16, wherein the transducer includes a piezoelectric transducer.

20. The system of claim 16, wherein the distal end of the shaft includes a hydrophilic coating.

21. The system of claim 16, wherein a proximal portion of the shaft of the guidewire includes a telescope mechanism movable between an expanded configuration and a contracted configuration.

22. The system of claim 21, wherein the telescope mechanism includes a plurality of telescoping elements, each of the telescoping elements configured to be sequentially received within an immediately adjacent one of the telescoping elements as the telescope mechanism is moved from the expanded configuration toward the contracted configuration.

23. The system of claim 21, wherein the telescope mechanism includes markings indicating a length of contraction of the guidewire.

24. The system of claim 16, wherein the electronic connector includes a PCBA board.

25. A guidewire device, comprising:
a shaft configured to be inserted through a channel of an endoscope to a target location within a duct anatomy, the shaft extending from a proximal end to a distal end including a transducer configured to emit an ultrasonic energy to a surrounding tissue.

26. The device of claim 25, wherein the transducer receives reflected echoes and converts them into electrical signals to generate an ultrasound image.

27. The device of claim 25, further comprising:
a rotator at the proximal end of the shaft, the rotator configured to rotate the transducer about a longitudinal axis of the device so that the transducer is configured to generate a 360-degree ultrasound image.

28. The device of claim 25, further comprising:
a telescope mechanism along a proximal portion of the shaft, the telescope mechanism movable between an expanded configuration and a contracted configuration to measure a length of contraction of the shaft.

29. The device of claim 28, wherein the telescope mechanism includes a plurality of telescoping elements, each of the telescoping elements configured to be sequentially received within an immediately adjacent one of the telescoping elements as the telescope mechanism is moved from the expanded configuration toward the contracted configuration.

30. The device of claim 25, wherein the shaft includes a plurality of the transducers extending along a distal portion thereof, the plurality of the transducer configured to deliver ultrasonic energy for dissolving a biliary stone.

31. A method for treating a duct anatomy during an endoscopic retrograde cholangiopancreatology, comprising:
inserting a guidewire through a channel of an endoscope to a target location within the duct anatomy and navigating the guidewire to the target location under ultrasound image guidance provided via a transducer at a distal end of the guidewire, which emits ultrasonic energy to a surrounding tissue to generate an ultrasound image.

32. The method of claim 31, further comprising:
positioning the distal end of the guidewire in alignment with a distal end of an obstruction within the target location of the duct anatomy; and
retracting a distal portion of the guidewire toward a proximal end of the guidewire until the distal end of the guidewire is in alignment with a proximal end of the obstruction within the target location to determine a length of the obstruction.

33. The method of claim 32, wherein retracting the distal portion of the guidewire toward the proximal end of the guidewire includes moving a telescope mechanism from an expanded configuration toward a contracted configuration.

34. The method of claim 33, wherein the telescope mechanism includes a plurality of telescoping elements configured to be sequentially received within an immediately adjacent one of the telescoping elements as the telescope mechanism is moved from the expanded configuration toward the contracted configuration.

35. The method of claim 31, wherein the transducer is rotatable about a longitudinal axis of the guidewire via a rotator at a proximal end of the guidewire to generate a 360-degree ultrasound image.
